(19) United States
(12) Patent Application Publication
(10) Pub. No.: US 2021/0128149 A1
(43) Pub. Date: May 6, 2021

(54) SURGICAL STAPLE CARTRIDGE

(71) Applicant: Covidien LP, Mansfield, MA (US)

(72) Inventors: Kenneth H. Whitfield, North Haven, CT (US); Thomas Casasanta, Southington, CT (US); Wojciech Kisiel, North Haven, CT (US)

(21) Appl. No.: 16/671,339

(22) Filed: Nov. 1, 2019

(51) Int. Cl. A61B 17/072; A61B 17/068

(52) U.S. Cl. CPC A61B 17/07207; A61B 17/0686; A61B 2017/07285; A61B 2017/07271; A61B 2017/07228

(57) ABSTRACT

A staple cartridge includes a cartridge body and a plurality of staples. The cartridge body defines a plurality of rows of staple pockets and a central knife slot. One of the staples is received in each of the staple pockets. Each of the staples defines a burr or facet that faces the central knife slot and defines a plane that is substantially parallel to the longitudinal axis of the cartridge body.

### FIELD

This disclosure is directed to a staple cartridge including spaced rows of staples, and more particularly, to a staple cartridge including spaced rows of staples that are adapted to deform in a predetermined manner.

### BACKGROUND

When a surgical stapling and dissecting device is operated to remove tissue from a patient, a knife of the device is advanced to dissect the tissue along a cut line. This creates a wound that is sealed with rows of staples that are ejected from the device and formed through the tissue along the cut line as the tissue is dissected. The staple rows seal the wound by providing pressure to the cut line to maintain hemostasis. Typically, the staples are aligned in rows on each side of the cut line. Each row of staples has one side that faces the cut line and another side that is exposed to blood pressure.

When the staples are deformed into tissue, tips of the staples may bend to the blood pressure side of the rows of staples. When this occurs, the tips of the staples may penetrate and perforate the tissue on the blood pressure side of the rows of staples which may result in oozing of blood and air from the perforations.

A continuing need exists for a staple cartridge that includes rows of staples that can be deformed in a manner to minimize perforations on the blood pressure side of the rows of staples.

### SUMMARY

One aspect of this disclosure is directed to a staple cartridge including a cartridge body and a plurality of staples, the cartridge body defines a longitudinal axis and has a plurality of staple pockets and a central knife slot. The staple pockets are arranged in at least one row on each side of the central knife slot. The plurality of staple pockets receives the plurality of staples. Each of the plurality of staples includes a back span and a leg extending from each end of the back span. Each of the legs includes a tip having a burr or facet that faces the central knife slot and defines a plane that extends in a direction substantially parallel to the longitudinal axis of the cartridge body.

In aspects of the disclosure, the at least one row includes a plurality of rows including an inner row and an outer row.

In some aspects of the disclosure, the staple cartridge includes a plurality of pushers supported with the plurality of staple pockets of the cartridge body, and each of the plurality of pushers supports at least one of the plurality of staples and is movable from a first position to a second position to eject the plurality of staples from the plurality of pockets of the cartridge body.

In certain aspects of the disclosure, the staple cartridge includes an actuation sled that is positioned and movable within the cartridge body to move the pushers from their first position to their second position.

In aspects of the disclosure, the cartridge body includes an upper clamping surface and a lower portion opposite to the upper clamping surface and the staple cartridge includes a guard that is supported on the lower portion of the cartridge body to retain the plurality of staples and the plurality of pushers within the cartridge body.

In some aspects of the disclosure, the guard is secured to the cartridge body by snap-fitting.

Another aspect of the disclosure is directed to a surgical stapling device including an elongate body and a tool assembly. The tool assembly is supported on the elongate body and includes an anvil and a cartridge assembly. The cartridge assembly includes a staple cartridge having a cartridge body and a plurality of staples. The cartridge body defines a longitudinal axis and has a plurality of staple pockets and a central knife slot. The staple pockets are arranged in at least one row on each side of the central knife slot. The plurality of staple pockets receives the plurality of staples. Each of the plurality of staples includes a back span and a leg extending from each end of the back span. Each of the legs includes a tip having a burr or facet that faces the central knife slot and defines a plane that extends in a direction that is substantially parallel to the longitudinal axis of the cartridge body.

In aspects of the disclosure, the stapling device includes a handle assembly and the elongate body is supported on and extends distally from the handle assembly.

In some aspects of the disclosure, the handle assembly is a battery powered handle assembly.

Yet another aspect of the disclosure is directed to a surgical stapling device that includes a handle assembly, an elongate body, and a tool assembly. The elongate body is supported on and extends distally from the handle assembly. The tool assembly is supported on the elongate body and includes an anvil and a cartridge assembly. The cartridge assembly includes a staple cartridge having a cartridge body, a plurality of staples, a plurality of pushers, and an actuation sled. The cartridge body defines a longitudinal axis and has a plurality of staple pockets and a central knife slot. The staple pockets include an inner row and an outer row of pockets on each side of the central knife slot. The plurality of staple pockets receives the plurality of staples. Each of the plurality of staples includes a back span and a leg extending from each end of the back span. Each of the legs includes a tip having a burr or facet that faces the central knife slot and defines a plane, wherein the plane extends in a direction substantially parallel to the longitudinal axis of the cartridge body. The plurality of pushers is supported within the plurality of staple pockets of the cartridge body. Each of the plurality of pushers supports at least one of the plurality of staples and is movable from a first position to a second position to eject the plurality of staples from the plurality of pockets of the cartridge body. The actuation sled is positioned within the cartridge body and is movable within the cartridge body to move the plurality of pushers from their first position to their second position.

Other features of the disclosure will be appreciated from the following description.

### BRIEF DESCRIPTION OF THE DRAWINGS

Various embodiments of the disclosed staple cartridge are described herein below with reference to the drawings, wherein:

FIG. 1 is a side perspective view of a surgical stapling device including an exemplary embodiment of the disclosed staple cartridge;

FIG. 2 is a side perspective view of the staple cartridge illustrated in FIG. 1;

FIG. 3 is an exploded perspective view of the staple cartridge shown in FIG. 2;

FIG. 4 is a side perspective view of the staple cartridge shown in FIG. 2 with all of the components of the staple cartridge shown in phantom except for the staples;

FIG. 5 is an enlarged view of the indicated area of detail shown in FIG. 3;

FIG. 6 is a cross-sectional view of the staple cartridge taken along section line 6-6 of FIG. 2;

FIG. 7 is a perspective view of a staple of the staple cartridge shown in FIG. 3 prior to formation;

FIG. 8 is a perspective view of the staple shown in FIG. 7 after formation;

FIG. 9 is a bottom view of the staple shown in FIG. 7 after formation; and

FIG. 10 is a top view tissue including the formed staples of FIG. 8.

### DETAILED DESCRIPTION

The disclosed surgical stapling device and staple cartridge will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure. As used herein, the terms “parallel”, “perpendicular”, and “aligned” are understood to include relative configurations that are substantially parallel, substantially perpendicular, and substantially aligned, i.e., up to about + or −10 degrees from true parallel, true perpendicular, true alignment.

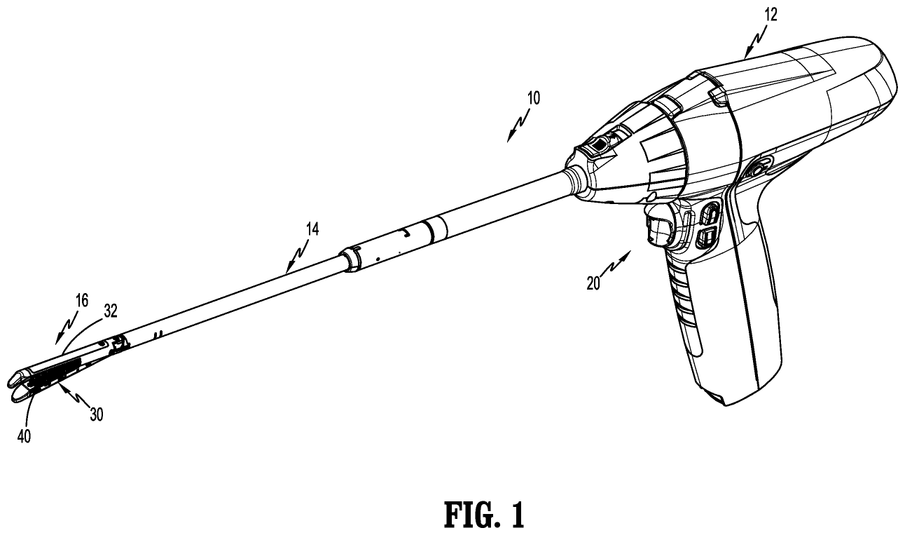

FIG. 1 illustrates a surgical stapling device shown generally as stapling device 10 that includes a handle assembly 12, an elongate body or adapter 14, and a tool assembly 16. As illustrated, the handle assembly 12 is powered and includes a stationary handgrip 18 and actuation buttons 20. The actuation buttons 20 are operable to actuate various functions of the tool assembly 16 via the adapter 14 including approximation, stapling, and dissection. In certain aspects of the disclosure, the handle assembly 16 supports batteries (not shown) that provide power to the handle assembly 12 to operate the stapling device 10. Although the stapling device 10 is illustrated as a powered stapling device, it is envisioned that the advantages of this disclosure are suitable for use with manually powered surgical stapling devices as well as robotically controlled stapling devices.

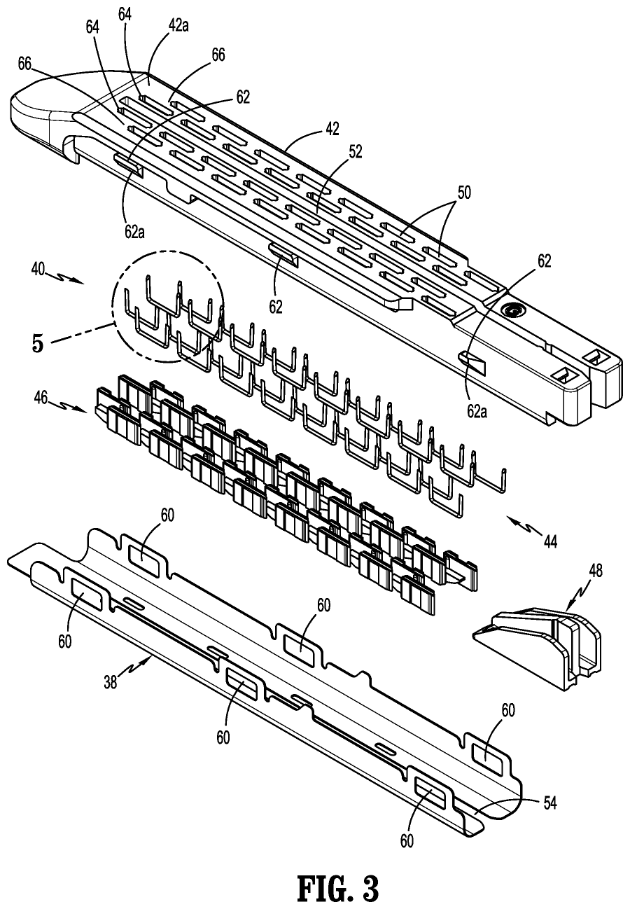

The tool assembly 16 of the stapling device 10 includes a cartridge assembly 30 and an anvil 32. The cartridge assembly 30 and the anvil 32 are coupled together such that the tool assembly 16 can pivot between an open position and a clamped position. The cartridge assembly 30 includes a guard 38 (FIG. 3) and a staple cartridge 40 including exemplary aspects of the disclosure.

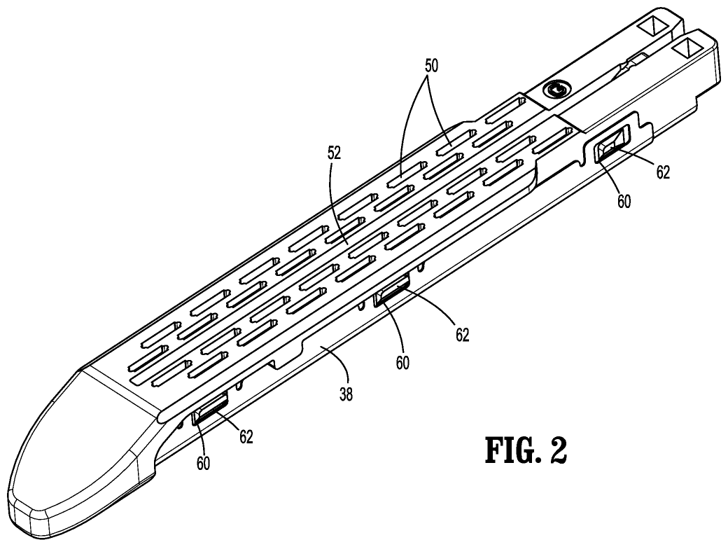

FIGS. 2-6 illustrate the staple cartridge 40 which includes a cartridge body 42, a plurality of staples 44, pushers 46, and an actuation sled 48. The cartridge body 42 includes an upper clamping surface 42a that defines a plurality of staple pockets 50 and a central knife slot 52 that extends along a midline of the cartridge body 42. As known in the art, the central knife slot 48 facilitates translation of a knife bar (not shown) through the tool assembly 16 to advance the actuation sled 48 through the cartridge body 42. The actuation sled 48 is positioned to engage the pushers 46 as the actuation sled 48 advances through the cartridge body 42 to drive the pushers 46 upwardly as viewed in FIG. 3 to drive the pushers 46 through the staple pockets 50. The staples 44 are supported on a respective pusher 46 and are driven from the staple pockets 50 as the pushers 46 are moved through the staple pockets 50. U.S. Pat. No. 5,865,361 discloses a stapling device including a knife bar that is movable through a tool assembly to eject staples from a staple cartridge and to cut tissue clamped between an anvil and a cartridge assembly of the stapling device. Although the cartridge body 42 is illustrated as being linear, it is envisioned that the cartridge body 42 may have a non-linear configuration along its longitudinal axis.

The staple guard 38 is secured to a lower side of the cartridge body 42 opposite to the upper clamping surface 42a and provides a cover to prevent the pushers 46 and the staples 44 from falling from the lower side of the cartridge body 42. In certain aspects of the disclosure, the guard includes openings 60 that receive projections 62 on the cartridge body 42 such that the guard 38 can be snap-fit onto the cartridge body 42. Each of the projections 62 can include an angled surface 62a that is angled outwardly towards the upper clamping surface 42a of the cartridge body 42. The angled surface 62a allows portions of the guard 38 to be cammed outwardly past the projections 62 as the guard 38 is attached to the cartridge body 42. It is envisioned that other securement techniques and devices can be used to secure the guard 38 to the cartridge body 42. The guard 38 defines a knife slot 44 that is aligned with the knife slot 52 formed in the cartridge body 42.

In aspects of the disclosure, the staple pockets 50 are arranged in rows that are positioned on each side of the central knife slot 52 of the cartridge body 42. Each of the staple pockets 50 receives a staple 44 such that the staples 44 are aligned in rows on each side of the central knife slot 50 within the cartridge body 42 of the staple cartridge 40. In some aspects of the disclosure, two rows of staples 44 are provided on each side of the central knife slot 50 including an inner row 64 and an outer row 66. It is envisioned that the cartridge body 42 may include one or more rows of staples 44 and staple pockets 50 on each side of the central knife slot 52, e.g., three rows or one row.

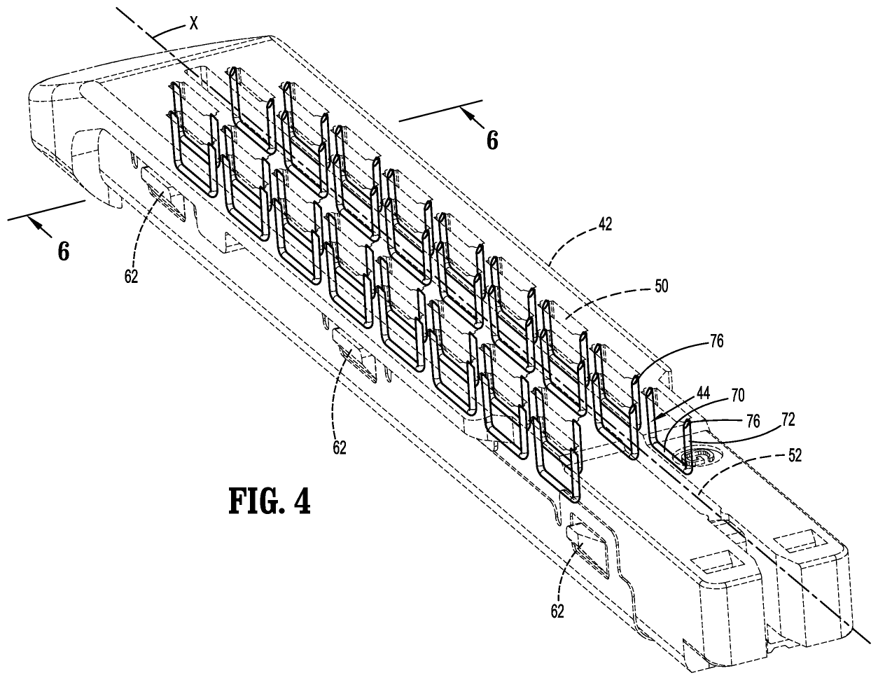

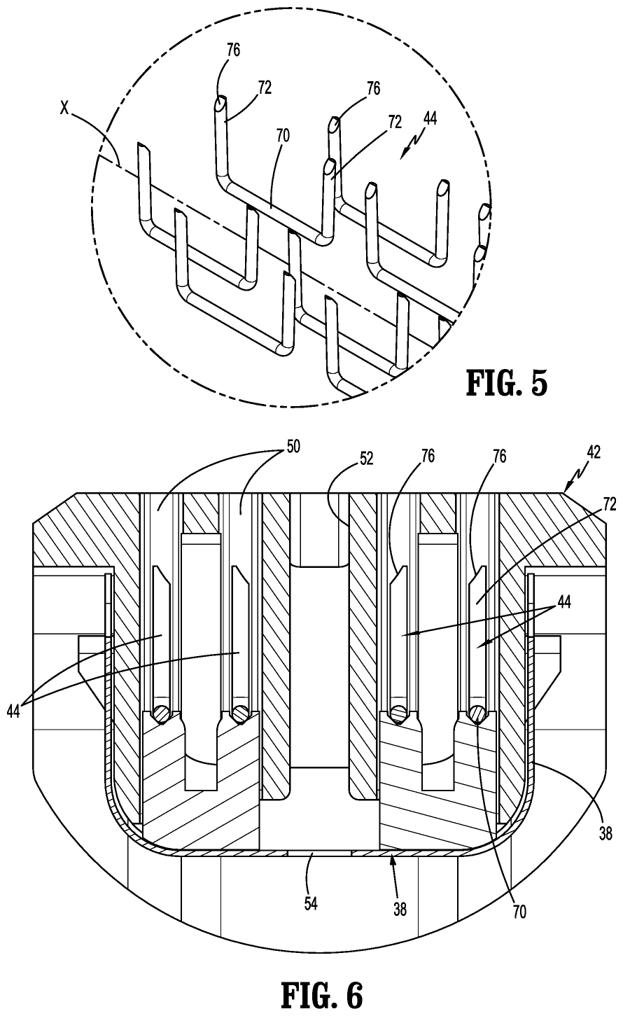

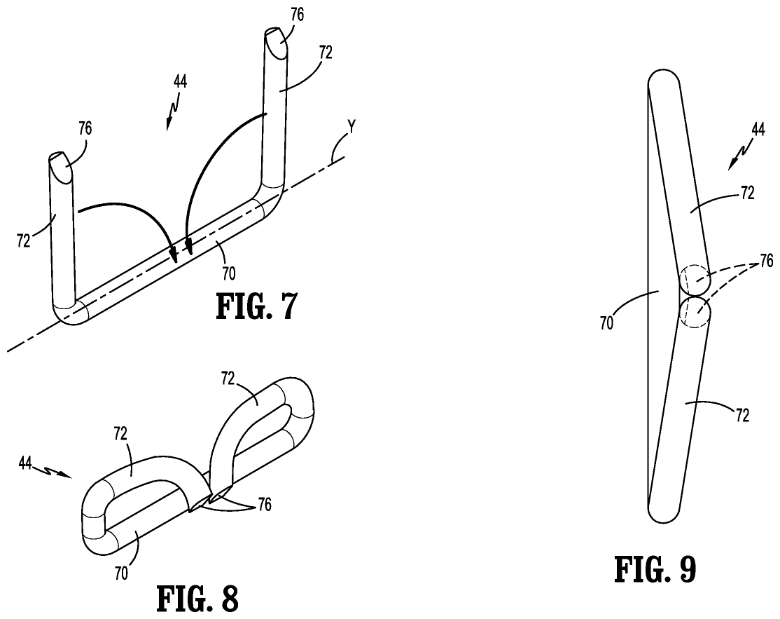

FIGS. 4-6 illustrate enlarged views of the staples 44 as they are supported within the cartridge body 42 of the cartridge assembly 40. The longitudinal axis “X” defined by the cartridge body 42 is shown in FIG. 5 extending through central knife slot 52 of the cartridge body 42. Each of the staples 44 includes a back span 70, and a leg 72 that extends from each end of the back span 70. Each of the legs 72 includes a tip 74 that is tapered to define a burr or facet forming an angled surface 76. In some aspects of the disclosure, the facet 76 defines a plane that extends in a direction that is substantially parallel to the longitudinal axis “X” of the cartridge body 42 and substantially parallel to a longitudinal axis “Y” (FIG. 7) defined by the back span 70 of the staple 44.

FIGS. 7-10 illustrate the configuration of the staples 44 prior to deformation (FIG. 7) and subsequent to deformation (FIGS. 8 and 9). Prior to deformation of the staples 44, the legs 72 of the staples 44 are substantially perpendicular to the longitudinal axis “Y” defined by the back span 70 of the staple 44. When the staples 44 are deformed into the anvil 32 of the tool assembly 16 (FIG. 1), the legs 72 are deformed to define a B-shaped configuration. Because of the orientation of the facets 76 on each of the legs 72 of the staples 44, the legs 72 of the staples 44 bend laterally from the longitudinal axis “Y” in the direction that the facet 76 is facing.

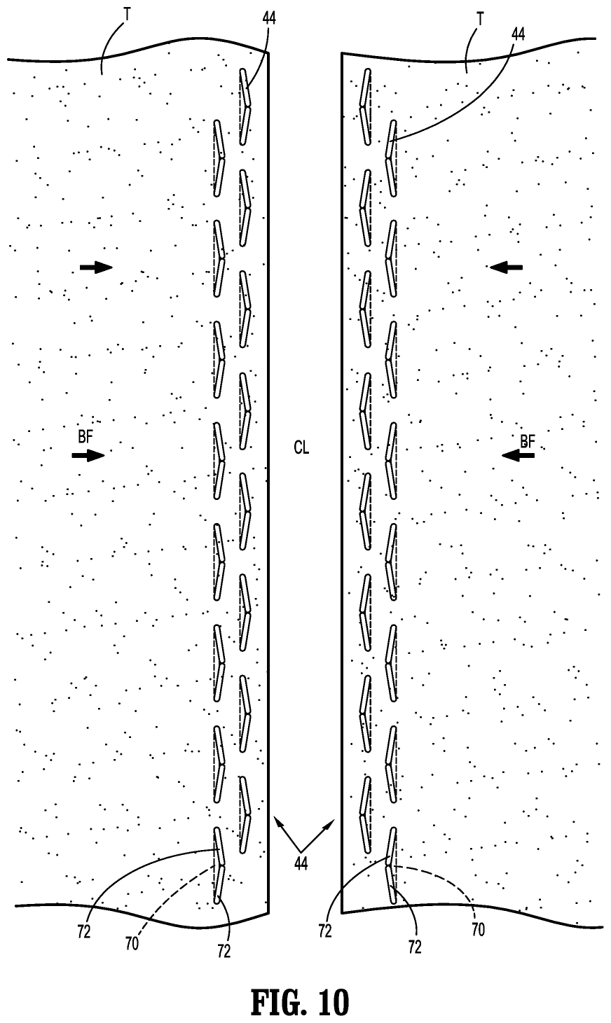

FIG. 10 illustrates staple placement within tissue “T” after the staples 44 have been formed in tissue. As discussed above, the staples 44 in each of the inner and outer rows 64 and 66 are supported within the staple pockets 50 of the cartridge body 42 with the facets 76 facing the cutline “CL”. As such, when the staples 44 are deformed in tissue “T” as shown in FIG. 10, the legs 72 of the staples 44 will move laterally in relation to the back span 70 towards the cutline “CL”. When this occurs, the tips 74 of the staples 44 will only penetrate tissue on the cutline “CL” side of the rows 64 and 66 of staples 44. After the tissue “T” is cut along the cutline “CL”, blood flow “BF” within the tissue “T” will only be in one direction “K” which is towards the cutline “CL”. As such, any perforations that may be formed in the tissue “T” during formation of the staples by the tips 74 of the staples 44 will be formed on the cutline “CL” side of the rows 64 and 66 of staples 44 rather than on the blood flow “BF” side of the rows 64 and 66 of staples 44. This will reduce the amount of blood that will communicate with any perforations that may have been formed by the tips 74 of the legs 72 of the staples 44 during staple formation to reduce the amount of oozing of blood from the stapled tissue “T”.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A staple cartridge comprising:
a cartridge body defining a longitudinal axis and having a plurality of staple pockets and a central knife slot, the staple pockets arranged in at least one row on each side of the central knife slot; and
a plurality of staples, each of the plurality of staple pockets receiving one of the plurality of staples, each of the plurality of staples including back span, and a leg extending from each end of the back span, each of the legs including a tip having a facet that faces the central knife slot and defines a plane, the plane extending in a direction substantially parallel to the longitudinal axis of the cartridge body.

2. The staple cartridge of claim 1, wherein the at least one row includes a plurality of rows, the plurality of rows including an inner row and an outer row.

3. The staple cartridge of claim 1, further including a plurality of pushers supported with the plurality of staple pockets of the cartridge body, each of the plurality of pushers supporting at least one of the plurality of staples and being movable from a first position to a second position to eject the plurality of staples from the plurality of pockets of the cartridge body.

4. The staple cartridge of claim 3, further including an actuation sled that is positioned within the cartridge body, the action sled being movable within the cartridge body to move the pushers from their first position to their second position.

5. The staple cartridge of claim 4, wherein cartridge body includes an upper clamping surface and a lower portion opposite to the upper clamping surface, the staple cartridge further including a guard supported on the lower portion of the cartridge body, the guard retaining the plurality of staples and the plurality of pushers within the cartridge body.

6. The staple cartridge of claim 5, wherein the guard is secured to the cartridge body by snap-fitting.

7. A surgical stapling device comprising:
an elongate body;
a tool assembly supported on the elongate body, the tool assembly including an anvil, and a cartridge assembly, the cartridge assembly including a staple cartridge, the staple cartridge including:
a cartridge body defining a longitudinal axis and having a plurality of staple pockets and a central knife slot, the staple pockets arranged in at least one row on each side of the central knife slot; and
a plurality of staples, each of the plurality of staple pockets receiving one of the plurality of staples, each of the plurality of staples including back span that extends in a direction substantially parallel to the longitudinal axis of the cartridge body, and a leg extending from each end of the back span, each of the legs including a tip having a facet that is oriented to face the central knife slot and define a plane, the plane extending in a direction substantially parallel to the longitudinal axis of the cartridge body, wherein the orientation of the facets on the legs of the staples cause all of the staple legs to be formed on a side of the back span toward the central knife slot.

8. The stapling device of claim 7, further including a handle assembly, the elongate body supported on and extending distally from the handle assembly.

9. The stapling device of claim 8, wherein the handle assembly is a battery powered handle assembly.

10. The stapling device of claim 8, wherein the at least one row includes a plurality of rows, the plurality of rows including an inner row and an outer row.

11. The stapling device of claim 7, further including a plurality of pushers supported with the plurality of staple pockets of the cartridge body, each of the plurality of pushers supporting at least one of the plurality of staples and being movable from a first position to a second position to eject the plurality of staples from the plurality of pockets of the cartridge body.

12. The stapling device of claim 11, further including an actuation sled that is positioned within the cartridge body, the action sled being movable within the cartridge body to move the plurality of pushers from their first position to their second position.

13. The stapling device of claim 12, wherein cartridge body includes an upper clamping surface and a lower portion opposite to the upper clamping surface, the staple cartridge further including a guard supported on the lower portion of the cartridge body, the guard retaining the plurality of staples and the plurality of pushers within the cartridge body.

14. The stapling device of claim 13, wherein the guard is secured to the cartridge body by snap-fitting.

15. A surgical stapling device comprising:
a handle assembly, the elongate body supported on and extending distally from the handle assembly.
an elongate body;
a tool assembly supported on the elongate body, the tool assembly including an anvil, and a cartridge assembly, the cartridge assembly including a staple cartridge, the staple cartridge including:
a cartridge body defining a longitudinal axis and having a plurality of staple pockets and a central knife slot, the staple pockets including an inner row and an outer row of pockets on each side of the central knife slot; and
a plurality of staples, each of the plurality of staple pockets receiving one of the plurality of staples, each of the plurality of staples including back span that extends in a direction substantially parallel to the longitudinal axis of the cartridge body, and a leg extending from each end of the back span, each of the legs including a tip having a facet that is oriented to face the central knife slot and define a plane, the plane extending in a direction substantially parallel to the longitudinal axis of the cartridge body, wherein the orientation of the facets on the legs of the staples cause all of the staple legs to be formed on a side of the back span toward the central knife slot;
a plurality of pushers supported within the plurality of staple pockets of the cartridge body, each of the plurality of pushers supporting at least one of the plurality of staples and being movable from a first position to a second position to eject the plurality of staples from the plurality of pockets of the cartridge body;
an actuation sled positioned within the cartridge body, the action sled being movable within the cartridge body to move the plurality of pushers from their first position to their second position.

16. The stapling device of claim 15, wherein cartridge body includes an upper clamping surface and a lower portion opposite to the upper clamping surface, the staple cartridge further including a guard supported on the lower portion of the cartridge body, the guard retaining the plurality of staples and the plurality of pushers within the cartridge body.

17. The stapling device of claim 16, wherein the guard is secured to the cartridge body by snap-fitting.
